(19) United States
(12) Patent Application Publication
(10) Pub. No.: US 2012/0214986 A1
(43) Pub. Date: Aug. 23, 2012

(54) PROCESS FOR THE PREPARATION OF IRON (III) CARBOXYMALTOSE COMPLEX

(75) Inventors: Dodda Mohan Rao, Hyderabad (IN); Pingili Krishna Reddy, Hyderabad (IN); Buthukuri Venkat Reddy, Hyderabad (IN)

(73) Assignee: SYMED LABS LIMITED, Hyderabad, Andhra Pradesh (IN)

(21) Appl. No.: 13/505,115

(22) PCT Filed: Nov. 4, 2009
(86) PCT No.: PCT/IN2009/000624

§ 371 Date: Apr. 30, 2012

(30) Foreign Application Priority Data
Nov. 4, 2009 (IN) PCT/IN2009/000624

(51) Int. Cl. C07H 23/00

(52) U.S. Cl. 536/121

(57) ABSTRACT

The present invention relates to novel processes for the preparation of iron (III) carboxymaltose complex. Thus, for example, a mixture of one or more maltodextrins, ferric hydroxide and water is heated, the resulting iron maltodextrin complex is oxidized using an aqueous sodium hypochlorite solution, followed by maintaining at 25° C. to 125° C. to produce iron (III) carboxymaltose complex.

### FIELD OF THE INVENTION

The present invention relates to novel processes for the preparation of iron (III) carboxymaltose complex.

### BACKGROUND OF THE INVENTION

Water-soluble iron carbohydrate complexes are used for the prophylaxis or treatment of iron deficiency anaemia. The medicaments are especially useful for parenteral application.

Ferric carboxymaltose complex, also known as iron (III) carboxymaltose, is indicated for the treatment of iron deficiency anaemia, and which is marketed by Vifor under the brand name Ferinject®.

U.S. Patent Application Publication No. 2006/0205691 discloses water-soluble iron carbohydrate complexes (ferric carboxymaltose complexes) obtainable from an aqueous solution of an iron (III) salt, preferably iron (III) chloride, and an aqueous solution of the oxidation product of one or more maltodextrins using an aqueous hypochlorite solution.

The process for the preparation of ferric carboxymaltose complex described in the aforementioned prior art suffers from disadvantages since it involves the use of excess amounts of sodium carbonate and iron (III) chloride, thereby generating a large quantity of chemical waste which is difficult to treat. Moreover, the use of highly corrosive and toxic reagents like iron (III) chloride and excess amounts of sodium carbonate in the final stage of complex formation is not advisable since the resulting complex is contaminated with unacceptable amounts of impurities like sodium carbonate and sodium chloride, and thus resulting in a poor product quality.

Similar process for the preparation of ferric carboxymaltose complex is also mentioned in PCT Publication No. WO 2007/081744. The '744 publication describes a method of treating iron-associated diseases comprising administering to a subject in need thereof an iron carbohydrate complex in a single dosage unit of at least about 0.6 grams of elemental iron, wherein the iron carbohydrate complex has a substantially non-immunogenic carbohydrate component and substantially no cross reactivity with anti-dextran antibodies.

Hence, the ferric carboxymaltose complex obtained by the process described in the prior art is not suitable for the preparation of medicaments without further purification.

Based on the aforementioned drawbacks, the prior art processes have been found to be unsuitable for the preparation of ferric carboxymaltose at lab scale and in commercial scale operations.

We have surprisingly discovered novel, improved and environmentally friendly processes for the preparation of iron (III) carboxymaltose complex substantially free of impurities using ferric hydroxide. The novel processes avoid the use of excess amounts of reagents like sodium carbonate and iron (III) chloride and thereby resolving the problems associated with the processes described in the prior art, and which are more convenient to operate at lab scale and in commercial scale operations. Desirable process properties include use of non-hazardous, environmentally friendly and easy to handle reagents in the final stage of complex formation, reduced quantities of reagents and base, reduced cost, greater simplicity, increased quality and increased yield of the product, and thereby enabling the commercially viable and environmentally friendly production of ferric carboxymaltose. The iron (III) carboxymaltose complexes obtained by the processes described herein can be directly used for the production of medicaments even without further purification.

### SUMMARY OF THE INVENTION

In one aspect of the present invention, there is provided a process for the preparation of water soluble iron (III) carboxymaltose complex on the basis of the oxidation products of maltodextrins, which comprises:
a. oxidizing one or more maltodextrins using an aqueous sodium hypochlorite solution to obtain a solution; and
b. reacting the aqueous solution obtained in step-(a) with ferric hydroxide to produce iron (III) carboxymaltose complex where, when one maltodextrin is applied, its dextrose equivalent lies between 4 and 20, and when a mixture of several maltodextrins is applied, the dextrose equivalent of the mixture lies between 4 and 20 and the dextrose equivalent of each individual maltodextrins contained in the mixture lies between 2 and 40.

In another aspect of the present invention, there is provided a process for the preparation of water soluble iron (III) carboxymaltose complex on the basis of the oxidation products of maltodextrins, which comprises adding sodium hypochlorite to a mixture of one or more maltodextrins and ferric hydroxide in the presence of water to obtain iron (III) carboxymaltose complex where, when one maltodextrin is applied, its dextrose equivalent lies between 4 and 20, and when a mixture of several maltodextrins is applied, the dextrose equivalent of the mixture lies between 4 and 20 and the dextrose equivalent of each individual maltodextrins contained in the mixture lies between 2 and 40.

In another aspect of the present invention, there is provided a process for the preparation of iron (III) carboxymaltose complex, which comprises:
a. heating a mixture of one or more maltodextrins, ferric hydroxide and water to obtain a iron maltodextrin complex; and
b. oxidizing the iron maltodextrin complex obtained in step-(a) using an aqueous sodium hypochlorite solution; and
c. maintaining the solution obtained in step-(b) at a temperature of 25° C. to 125° C. and at a pH of 5 to 12 to produce iron (III) carboxymaltose complex,

wherein, when one maltodextrin is applied, its dextrose equivalent lies between 4 and 20, and when a mixture of several maltodextrins is applied, the dextrose equivalent of the mixture lies between 4 and 20 and the dextrose equivalent of each individual maltodextrins contained in the mixture lies between 2 and 40.

### DETAILED DESCRIPTION OF THE INVENTION

According to one aspect of the present invention, there is provided a process for the preparation of water soluble iron (III) carboxymaltose complex on the basis of the oxidation products of maltodextrins, which comprises:
a. oxidizing one or more maltodextrins using an aqueous sodium hypochlorite solution to obtain a solution; and
b. reacting the aqueous solution obtained in step-(a) with ferric hydroxide to obtain produce iron (III) carboxymaltose complex wherein, when one maltodextrin is applied, its dextrose equivalent lies between 4 and 20, and when a mixture of several maltodextrins is applied, the dextrose equivalent of the mixture lies between 4 and 20 and the dextrose equivalent of each individual maltodextrins contained in the mixture lies between 2 and 40.

In order to prepare the liginds of the complex of the invention, the maltodextrins are oxidized in an aqueous solution with a sodium hypochlorite solution.

In one embodiment, the oxidation reaction in step-(a) is carried out in an alkaline solution at a pH of 8 to 12. In another embodiment, the oxidation reaction is carried out at a temperature of 15 to 40° C., and preferably at a temperature of 25 to 35° C. In another embodiment, the oxidation reaction is carried out for 10 minutes to 4 hours, and preferably for 1 hour to 1 hour 30 minutes.

Optionally, the oxidation reaction is carried out in the presence of a catalyst such as alkali bromides, for example sodium bromide. The amount of catalyst is not critical. Specifically, the amount of catalyst is kept as low as possible in order to achieve an end product (Fe-complex) which can easily be purified, and more specifically catalytic amounts are sufficient.

In order to prepare the complex of the invention, the obtained oxidized maltodextrins are reacted with ferric hydroxide. In order to do so, the oxidized maltodextrins can be isolated and re-dissolved. However, it is also possible to use the obtained aqueous solutions of the oxidized maltodextrins directly for the further reaction with ferric hydroxide. For instance, the aqueous solution of the oxidized maltodextrin can be mixed with ferric hydroxide in order to carry out the reaction.

Preferably, a freshly prepared ferric hydroxide is used in step-(b).

In one embodiment, the reaction in step-(b) is carried out at a pH between 5 and 14. If necessary, the pH of the reaction is adjusted with a strong base. Examples of strong bases are alkali or alkaline earth metal hydroxides such as sodium hydroxide.

In another embodiment, the reaction in step-(b) is carried out at a temperature of 15° C. to 125° C. It is preferred to raise the temperature gradually. Thus, for example, it is preferable to heat the reaction mixture at a temperature of about 15 to 70° C. and then raise the temperature gradually up to 125° C.

Optionally, the reaction is carried out under pressure.

The reaction is preferably carried out for 15 minutes to 4 hours depending on the reaction conditions.

In a preferred embodiment, the reaction in step-(b) is initially maintained at a pH of 10 to 12 and at a temperature of 40 to 60° C., followed by adjusting the pH between 4 to 7 and maintaining the reaction at a temperature of 85° C. to 125° C.

The pH can be lowered, if necessary, by the addition of an acid. It is preferable to use inorganic or organic acids or a mixture thereof, specifically hydrohalic acids such as aqueous hydrochloric acid.

After completion of the reaction, the obtained solution can be cooled to room temperature and then optionally diluted and filtered. After cooling, the pH is adjusted to 5 to 7 by the addition of an acid or a base. It is preferable to use the acids and bases which have been mentioned for carrying out the reaction. The solutions obtained are purified and can directly be used for the production of medicaments. However, it is also possible to isolate the iron (III) complex from the solution by precipitation with an alcohol such as an alkanol, for example, ethanol.

According to another aspect of the present invention, there is provided a process for the preparation of water soluble iron (III) carboxymaltose complex on the basis of the oxidation products of maltodextrins, which comprises adding sodium hypochlorite to a mixture of one or more maltodextrins and ferric hydroxide in the presence of water to produce iron (III) carboxymaltose complex wherein, when one maltodextrin is applied, its dextrose equivalent lies between 4 and 20, and when a mixture of several maltodextrins is applied, the dextrose equivalent of the mixture lies between 4 and 20 and the dextrose equivalent of each individual maltodextrins contained in the mixture lies between 2 and 40.

In order to prepare the iron (III) carboxymaltose complex of the invention, the mixture of maltodextrins and ferric hydroxide in an aqueous solution is oxidized with a sodium hypochlorite solution.

Preferably, a freshly prepared ferric hydroxide is used.

In one embodiment, the oxidation reaction is carried out in an alkaline solution at a pH of 8 to 12. In another embodiment, the oxidation reaction is carried out at a temperature of 15 to 40° C., and preferably at a temperature of 25 to 35° C. In another embodiment, the oxidation is carried out for 10 minutes to 4 hours and preferably 1 hour to 1 hour 30 minutes.

Optionally, the oxidation reaction is carried out in the presence of a catalyst such as alkali bromide, for example sodium bromide. The amount of catalyst is not critical. In one embodiment, the amount of the catalyst is kept as low as possible in order to achieve an end product (Fe-complex) which can easily be purified, and specifically catalytic amounts are sufficient.

In one embodiment, the pH of the reaction is raised to values in between 5 to 14 using a strong base. Examples of strong bases are alkali- or alkaline earth metal hydroxides such as sodium hydroxide.

In one embodiment, the reaction is carried out at a temperature of 15° C. to 125° C. It is preferred to raise the temperature gradually. Thus, for example, it is preferable to heat the reaction mixture at a temperature of about 15 to 70° C. and then raise the temperature gradually up to 125° C.

Optionally, the reaction is carried out under pressure.

The reaction is preferably carried out for 15 minutes to 4 hours depending on the reaction conditions.

In a preferred embodiment, the reaction is initially maintained at a pH of 10 to 12 and at a temperature of 40 to 60° C., followed by adjusting the pH at 4 to 7 and maintaining the reaction at a temperature of 85° C. to 125° C.

The pH can be lowered, if necessary, by the addition of an acid. It is preferable to use inorganic or organic acids or a mixture thereof, specifically hydrohalic acids such as aqueous hydrochloric acid.

After completion of the reaction, the obtained solution can be cooled to room temperature and then optionally diluted and filtered. After cooling, the pH is adjusted 5 to 7 by the addition of an acid or a base. It is preferable to use the acids and bases which have been mentioned for carrying out the reaction. The solutions obtained are purified and can directly be used for the production of medicaments. However, it is also possible to isolate the iron (III) complex from the solution by precipitation with an alcohol such as an alkanol, for example, ethanol.

According to another aspect of the present invention, there is provided a process for the preparation of iron (III) carboxymaltose complex, which comprises:
a. heating a mixture of one or more maltodextrins, ferric hydroxide and water to produce an iron maltodextrin complex;
b. oxidizing the iron maltodextrin complex obtained in step-(a) using an aqueous sodium hypochlorite solution; and
c. maintaining the solution obtained in step-(b) at a temperature of 25° C. to 125° C. and at a pH of 5 to 12 to produce iron (III) carboxymaltose complex,

wherein, when one maltodextrin is applied, its dextrose equivalent lies between 4 and 20, and when a mixture of several maltodextrins is applied, the dextrose equivalent of the mixture lies between 4 and 20 and the dextrose equivalent of each individual maltodextrins contained in the mixture lies between 2 and 40.

In one embodiment, the heating in step-(a) is carried out at a temperature of 50° C. to the boiling point while maintaining the pH between 5 and 14. Preferably, the heating is carried out at a temperature of 70 to 90° C., and more preferably at a temperature of about 90° C.

Preferably, a freshly prepared ferric hydroxide is used in step-(a).

In one embodiment, the oxidation reaction in step-(b) is carried out in an alkaline solution at a pH of 8 to 12. In another embodiment, the oxidation reaction is carried out at a temperature of 15 to 40° C. and preferably at 25 to 35° C. In another embodiment, the oxidation reaction is carried out for 10 minutes to 4 hours and preferably for 1 hour to 1 hour 30 minutes.

Optionally, the oxidation is carried out in presence of a catalyst such as alkali bromide, for example sodium bromide. The amount of catalyst is not critical. In one embodiment, the amount is kept as low as possible in order to achieve an end product (Fe-complex) which can easily be purified, and specifically catalytic amounts are sufficient.

In one embodiment, the pH of reaction in step-(c) is adjusted with a strong base. Examples of strong bases are alkali- or alkaline earth metal hydroxides such as sodium hydroxide.

In another embodiment, the reaction in step-(c) is carried out at a temperature of 25° C. to 125° C. It is preferred to raise the temperature gradually. Thus, for example, it is preferable to heat the reaction mixture at a temperature of about 25 to 70° C. and then raise the temperature gradually up to 125° C.

Optionally, the reaction is carried out under pressure.

The reaction is preferably carried out for 15 minutes to 4 hours depending on the reaction conditions.

In a preferred embodiment, the reaction in step-(c) is initially maintained at a pH of 10 to 12 and at a temperature of 40 to 60° C., followed by adjusting the pH between 4 to 7 and maintaining the reaction at a temperature of 85° C. to 125° C.

The pH can be lowered, if necessary, by the addition of an acid. It is preferable to use inorganic or organic acids or a mixture thereof, specifically hydrohalic acids such as aqueous hydrochloric acid.

After completion of the reaction, the obtained solution can be cooled to room temperature and then optionally diluted and filtered. After cooling, the pH is adjusted to 5 to 7 by the addition of an acid or a base. It is preferable to use the acids and bases which have been mentioned for carrying out the reaction. The solutions obtained are purified and can directly be used for the production of medicaments. However, it is also possible to isolate the iron (III) complex from the solution by precipitation with an alcohol such as an alkanol, for example, ethanol.

According to the processes of the present invention, the iron content of the obtained iron (III) carboxymaltose complexes is 10 to 40% weight/weight, and specifically 20 to 35% weight/weight. Iron content is measured by using Atomic Absorption Spectrophotometer (AAS). The complexes can easily be dissolved in water. It is possible to prepare neutral aqueous solutions which have an iron content of 1% weight/volume to 20% weight/volume. Such solutions can be sterilised by general methods. The weight average molecular weight of the obtained complexes is in between 80 kDa to 700 kDa, preferably 80 kDa to 350 kDa, and more preferably up to 300 kDa. The weight average molecular weight of the complexes is measured by the following method:

### Mobile Phase:

Weigh accurately about 3.56 g of sodium phosphate dihydrate, 2.76 g of monobasic sodium phosphate and 0.20 g of sodium azide in to a 1 liter standard volumetric flask containing 800 ml water.
Mix and dissolve the contents.
Make up to the mark with water.

### System Suitability Solution:—

Weigh accurately about 200.0 mg of high molecular weight dextran (Mw value 10,00,000 Da to 20,00,000 Da and Mw/Mn value 1.0 to 1.8) and 100.0 mg of glucose in to a 20 ml volumetric flask and make up to the volume with mobile phase.
   Standard solution-1:—
Weigh accurately about 20.0 mg of 5900 Da molecular weight standard in to a 5 ml volumetric flask.
Add 4 ml of mobile phase.
   Standard solution-2:—
Weigh accurately about 20.0 mg of 11,800 Da molecular weight standard in to a 5 ml volumetric flask
Add 4 ml of mobile phase.
   Standard solution-3:—
Weigh accurately about 20.0 mg of 22,800 Da molecular weight standard in to a 5 ml volumetric flask
Add 4 ml of mobile phase.
   Standard solution-4:—
Weigh accurately about 20.0 mg of 47,300 Da molecular weight standard in to a 5 ml volumetric flask
Add 4 ml of mobile phase.
   Standard solution-5:—
Weigh accurately about 20.0 mg of 112,000 Da molecular weight standard in to a 5 ml volumetric flask
Add 4 ml of mobile phase.
   Standard solution-6:—
Weigh accurately about 20.0 mg of 212,000 Da molecular weight standard in to a 5 ml volumetric flask
Add 4 ml of mobile phase.
   Standard solution-7:—
Weigh accurately about 20.0 mg of 404,000 Da molecular weight standard in to a 5 ml volumetric flask
Add 4 ml of mobile phase.

Allow each standard solution to stand at or below 25° C. for a minimum of 12 hours.

After the agglomerate particles of each standard solution have swelled to their fullest extent, gently swirl each standard solution until dissolved.

### Sample Preparation:

Transfer 5.0 ml of iron carboxymaltose complex in to a 10-ml volumetric flask.
Make up to the volume with mobile phase.

### Chromatographic System:

| | |
|---|---|
| Detector | Refractive index detector (RID) |
| Cell temperature | 45° C. |
| Columns | Waters, Ultrahydrogel 7.8-mmX30-cm 1000 A⁰ |
| | Waters, Ultrahydrogel 7.8-mmX30-cm 120 A⁰ |
| Columns temperatures | 45 ± 2° C. |
| Flow rate | 0.5 mL per minute. |
| Run Time | 50 min. |
| GPC software | GPC for Class-VP 1.02 version |

### System Suitability:

Equilibrate the columns and system for 1 hour.
Inject exactly 25 μL of mobile phase in to the system and record the chromatogram to a run time of 50 minutes as a blank.
Program the data processor to inhibit the peaks due to blank.
Inject exactly 25 μL of system suitability solution in to the system and record the chromatogram to a run time of 50 minutes.
Calculate the resolution between high molecular weight dextran and glucose.

### Requirement:

The resolution between high molecular weight dextran and glucose should not be less than 4.0.

### Procedure:

Inject exactly 25 μL of each standard solution separately and record the chromatograms to a run time of 50 minutes.
Inject exactly 25 μL of Iron carboxymaltose complex and record the chromatogram.
Enter the retention times and Molecular weights of standard solutions in the GPC software to generate a calibration curve.
Calculate the correlation coefficient of calibration curve.
   Note: Correlation coefficient of calibration curve should not be less than 0.98.
Superimpose the chromatogram of the Iron carboxymaltose complex on the calibration curve.
Record the weight average molecular weight (Mw).

The following examples are given for the purpose of illustrating the present invention and should not be considered as limitations on the scope and spirit of the invention.

### EXAMPLES

### Example 1

Ferric chloride (61.5 gm) was dissolved in water (750 ml) and the solution was filtered to remove undissolved material. The resulting solution was cooled to 5 to 10° C. Sodium carbonate solution (61.5 gm dissolved in 750 ml water) was slowly added to the above solution at 5 to 10° C. The reaction mixture was stirred for 10 minutes at 5 to 10° C. and then again stirred for 1 hour at ambient temperature. The separated solid was filtered and washed with water to obtain ferric hydroxide.

### Example 2

Maltodextrin (50 gm, 14.2 dextrose equivalent), water (100 ml) and sodium bromide (0.35 gm) were added to a sodium hypochlorite solution (68 gm) containing 2.79 gm of active chlorine. The resulting solution was stirred for 5 minutes at ambient temperature and then the pH of the reaction mass was adjusted to 11 with dilute sodium hydroxide solution, and maintained for 1 hour 30 minutes. The resulting mass was added to a mixture of freshly prepared ferric hydroxide (obtained in example 1 from 61.5 g of ferric chloride) and water (100 ml). The pH of the reaction mass was then adjusted to 11 with dilute sodium hydroxide solution. The resulting mixture was heated to 50° C. and maintained for 30 minutes, and then the pH of the reaction mass was adjusted to 6 with dilute hydrochloric acid. The reaction mass was maintained for 30 minutes at 50° C., followed by maintaining for 30 minutes at 96 to 98° C. The resulting solution was cooled to ambient temperature to produce iron carboxymaltose complex.

Molecular Weight=3,00,000 Da

### Example 2a

Iron carboxymaltose solution (50 gm, obtained in example 2) was added to ethanol (1400 ml) and then stirred for 3 hours at ambient temperature. The resulting solid was collected by filtration, washed with ethanol and the solid was dried at 50° C. under vacuum for 2 hours to obtain iron carboxymaltose powder.

| | | |
|---|---|---|
| Molecular Weight = | 2,96,580 | Da |
| Iron content = | 21% | W/W |

### Example 3

Example 2 was repeated using maltodextrin (6 dextrose equivalent) instead of maltodextrin (14.2 dextrose equivalent) to obtain iron carboxymaltose complex.

| | | |
|---|---|---|
| Molecular Weight = | 2,58,000 | Da |
| Iron content = | 25 | mg/gm |

### Example 4

Example 2 was repeated using a mixture of maltodextrin (6 dextrose equivalent, 24 gm) and maltodextrin (14.2 dextrose equivalent, 28 gm) instead of maltodextrin (14.2 dextrose equivalent) to obtain iron carboxymaltose complex.

Molecular Weight=1,76,597 Da

About 50 percent of the volume was distilled from the above solution under vacuum to obtain iron carboxymaltose complex.

| | |
|---|---|
| Iron content = | 50 mg/ml |

### Example 5

Example 2 was repeated using maltodextrin (16 dextrose equivalent) instead of maltodextrin (14.2 dextrose equivalent) to obtain iron carboxymaltose complex.

| | | |
|---|---|---|
| Molecular Weight = | 2,50,800 | Da |
| Iron content = | 24 | mg/gm |

### Example 6

A solution of maltodextrin (50 gm, 14.2 dextrose equivalent) in water (100 ml) was added to a mixture of ferric hydroxide (obtained in example 1 from 61.5 gm of ferric chloride) and water (100 ml). The pH of the resulting mixture was adjusted to 11 with dilute sodium hydroxide solution, followed by the addition of sodium hypochlorite solution (70 gm) containing 3.1 gm of active chlorine and sodium bromide (0.5 gm) at ambient temperature. The pH of the reaction mixture was adjusted to 11 and then stirred for 1 hour at ambient temperature. The mixture was heated to 50° C. and maintained for 30 minutes. The pH of the resulting mass was adjusted to 6 with diluted hydrochloric acid and maintained for 30 minutes at 50° C. The reaction mass was heated at 96 to 98° C. and maintained for 1 hour. The solution was cooled to ambient temperature to produce iron carboxymaltose complex.

| | |
|---|---|
| Molecular Weight = | 2,78,000 Da |

### Example 6a

The iron carboxymaltose complex obtained in example 6 was precipitated by using ethanol. Iron carboxymaltose solution (50 gm, obtained in example 6) was added to ethanol (1400 ml) and then stirred for 3 hours at ambient temperature. The resulting solid was collected by filtration, washed with ethanol and then dried at 50° C. under vacuum for 2 hours to obtain iron carboxymaltose powder.

| | | |
|---|---|---|
| Molecular Weight = | 2,73,000 | Da |
| Iron content = | 22% | W/W |

### Example 7

Example 6 was repeated using maltodextrin (6 dextrose equivalent) instead of maltodextrin (14.2 dextrose equivalent) to obtain iron carboxymaltose complex.

| | | |
|---|---|---|
| Molecular Weight = | 3,10,000 | Da |
| Iron content = | 25 | mg/gm |

### Example 8

Example 6 was repeated using a mixture of maltodextrin (6 dextrose equivalent, 24 gm) and maltodextrin (14.2 dextrose equivalent, 28 gm) instead of maltodextrin (14.2 dextrose equivalent) to obtain iron carboxymaltose complex.

| | |
|---|---|
| Molecular Weight = | 1,38,000 Da |

About 50 percent of the volume was distilled from the above solution under vacuum to obtain iron carboxymaltose complex.

| | |
|---|---|
| Iron content = | 50 mg/ml |

### Example 9

Example 6 was repeated using maltodextrin (16 dextrose equivalent) instead of maltodextrin (14.2 dextrose equivalent) to obtain iron carboxymaltose complex.

| | | |
|---|---|---|
| Molecular Weight = | 4,55,919 | Da |
| Iron content = | 23.5 | mg/gm |

### Example 10

A solution of maltodextrin (50 gm, 14.2 dextrose equivalent) in water (100 ml) was added to a mixture of ferric hydroxide (obtained in example 1 from 61.5 gm of ferric chloride) and water (100 ml). The pH of the resulting mixture was adjusted to 11 with dilute sodium hydroxide solution, followed by the addition of sodium hypochlorite solution (70 gm) containing 3.1 gm of active chlorine and sodium bromide (0.5 gm). The pH of the reaction mixture was adjusted to 11 and stirred for 1 hour at ambient temperature. The mixture was heated to 50° C. and maintained for 30 minutes. The pH of the resulting mass was adjusted to 6 with dilute hydrochloric acid and maintained for 30 minutes. The mixture was taken in autoclave and maintained for 1 hour at 121° C. under 1.1 kg/cm² pressure. The solution was cooled to ambient temperature to produce iron carboxymaltose complex.

| | |
|---|---|
| Molecular Weight = | 2,28,000 Da |

The above solution was precipitated by using ethanol. Iron carboxymaltose solution (50 gm, obtained above) was added to ethanol (1400 ml) and then stirred for 3 hours at ambient temperature. The solid obtained was collected by filtration, washed with ethanol and then dried at 50° C. under vacuum for 2 hours to obtain iron carboxymaltose powder.

| | | |
|---|---|---|
| Molecular Weight = | 2,25,000 | Da |
| Iron content = | 21% | W/W |

### Example 11

Example 10 was repeated using maltodextrin (6 dextrose equivalent) instead of maltodextrin (14.2 dextrose equivalent) to obtain iron carboxymaltose complex.

| | | |
|---|---|---|
| Molecular Weight = | 2,80,000 | Da |
| Iron content = | 22.3 | mg/gm |

### Example 12

Example 10 was repeated using a mixture of maltodextrin (6 dextrose equivalent, 24 gm) and maltodextrin (14.2 dextrose equivalent, 28 gm) instead of maltodextrin (14.2 dextrose equivalent) to obtain iron carboxymaltose complex.

| | | |
|---|---|---|
| Molecular Weight = | 2,41,172 | Da |
| Iron content = | 23 | mg/gm |

### Example 13

Example 10 was repeated using maltodextrin (16 dextrose equivalent) instead of maltodextrin (14.2 dextrose equivalent) to obtain iron carboxymaltose complex.

| | | |
|---|---|---|
| Molecular Weight = | 3,10,000 | Da |
| Iron content = | 24.5 | mg/gm |

### Example 14

A solution of maltodextrin (50 gm, 14.2 dextrose equivalent) in water (100 ml) was added to a mixture of ferric hydroxide (obtained in example 1 from 61.5 gm of ferric chloride) and water (100 ml), followed by adjusting the pH to 11 with dilute sodium hydroxide solution. The reaction mixture was heated to 90° C. and maintained for 30 minutes at 90° C. The solution was cooled to ambient temperature to produce iron maltodextrin complex (Molecular weight: 2,40,000 Da).

Sodium hypochlorite solution (70 gm) containing 3.1 gm of active chlorine and sodium bromide (0.5 gm) were added to the iron (III) maltodextrin complex obtained above and then the pH was adjusted to 11. The solution was maintained for 1 hour at ambient temperature and heated to 50° C., and then maintained for 30 minutes. The pH was adjusted to 6 with dilute hydrochloric acid and maintained for 30 minutes at 50° C. The reaction mass was heated to 96 to 98° C. and maintained for 30 minutes. The solution was cooled to ambient temperature to produce iron carboxymaltose complex.

| | |
|---|---|
| Molecular Weight = | 1,90,000 Da |

### Example 14a

Iron carboxymaltose solution (50 gm, obtained in example 14) was added to ethanol (1400 ml) and then stirred for 3 hours at ambient temperature. The solid obtained was collected by filtration, washed with ethanol and then dried at 50° C. under vacuum for 2 hours to produce iron carboxymaltose powder.

| | | |
|---|---|---|
| Molecular Weight = | 1,88,000 | Da |
| Iron content = | 20% | W/W |

### Example 15

Example 14 was repeated using maltodextrin (6 dextrose equivalent) instead of maltodextrin (14.2 dextrose equivalent) to obtain iron carboxymaltose complex.

| | | |
|---|---|---|
| Iron maltodextrin complex molecular weight = | 5,53,240 | Da |
| Iron carboxymaltose complex molecular weight = | 3,40,000 | Da |
| Iron content = | 24.8 | mg/gm |

### Example 16

Example 14 was repeated using a mixture of maltodextrin (6 dextrose equivalent, 24 gm) and maltodextrin (14.2 dextrose equivalent, 28 gm) instead of maltodextrin (14.2 dextrose equivalent) to obtain iron carboxymaltose complex.

| | | |
|---|---|---|
| Iron maltodextrin complex molecular weight = | 2,55,569 | Da |
| Iron carboxymaltose complex molecular weight = | 2,39,612 | Da |

About 50 percent of the volume was distilled from the above solution under vacuum to obtain iron carboxymaltose complex.

| | |
|---|---|
| Iron content = | 50 mg/ml |

### Example 17

Example 14 was repeated using maltodextrin (16 dextrose equivalent) instead of maltodextrin (14.2 dextrose equivalent) to obtain iron carboxymaltose complex.

| | | |
|---|---|---|
| Molecular Weight = | 3,70,000 | Da |
| Iron content = | 23 | mg/gm |

1. A process for the preparation of water soluble iron (III) carboxymaltose complex on the basis of the oxidation products of maltodextrins, which comprises:
a) oxidizing one or more maltodextrins using an aqueous sodium hypochlorite solution to obtain a solution; and
b) reacting the aqueous solution obtained in step (a) with ferric hydroxide to produce iron (III) carboxymaltose complex wherein, when one maltodextrin is applied, its dextrose equivalent lies between 4 and 20, and when a mixture of several maltodextrins is applied, the dextrose equivalent of the mixture lies between 4 and 20 and the dextrose equivalent of each individual maltodextrins contained in the mixture lies between 2 and 40.

2. The process of claim 1, wherein the oxidation reaction in step-(a) is carried out in an alkaline solution at a pH of 8 to 12.

3. The process of claim 1, wherein the oxidation reaction in step-(a) is carried out at a temperature of 15 to 40° C.

4. The process of claim 1, wherein the oxidation reaction in step-(a) is carried out in the presence of sodium bromide.

5. The process of claim 1, wherein the reaction in step-(b) is carried out at a temperature of 15° C. to 125° C.

6. A process for the preparation of water soluble iron (III) carboxymaltose complex on the basis of the oxidation products of maltodextrins, which comprises adding sodium hypochlorite to a mixture of one or more maltodextrins and ferric hydroxide in the presence of water to produce iron (III) carboxymaltose complex wherein, when one maltodextrin is applied, its dextrose equivalent lies between 4 and 20, and when a mixture of several maltodextrins is applied, the dextrose equivalent of the mixture lies between 4 and 20 and the dextrose equivalent of each individual maltodextrins contained in the mixture lies between 2 and 40.

7. The process of claim 6, wherein the reaction is carried out in an alkaline solution at a pH of 8 to 12.

8. The process of claim 6, wherein the reaction is carried out in the presence of sodium bromide.

9. The process of claim 6, wherein the reaction is carried out at a temperature of 15° C. to 125° C.

10. A process for the preparation of iron (III) carboxymaltose complex, which comprises:
a) heating a mixture of one or more maltodextrins, ferric hydroxide and water to produce an iron maltodextrin complex;
b) oxidizing the iron maltodextrin complex obtained in step-(a) using an aqueous sodium hypochlorite solution; and
c) maintaining the solution obtained in step-(b) at a temperature of 25° C. to 125° C. and at a pH of 5 to 12 to produce iron (III) carboxymaltose complex;
wherein, when one maltodextrin is applied, its dextrose equivalent lies between 4 and 20, and when a mixture of several maltodextrins is applied, the dextrose equivalent of the mixture lies between 4 and 20 and the dextrose equivalent of each individual maltodextrins contained in the mixture lies between 2 and 40.

11. The process of claim 10, wherein the oxidation reaction in step-(b) is carried out in an alkaline solution at a pH of 8 to 12.

12. The process of claim 10, wherein the oxidation reaction in step-(b) is carried out at a temperature of 15 to 40° C.

13. The process of claim 10, wherein the oxidation reaction in step-(b) is carried out in the presence of sodium bromide.

14. The process of claim 1, wherein the iron content of the resulting iron (III) carboxymaltose complex is 10 to 40% W/W.

15. The process of claim 6, wherein the iron content of the resulting iron (III) carboxymaltose complex is 10 to 40% W/W.

16. The process of claim 10, wherein the iron content of the resulting iron (III) carboxymaltose complex is 10 to 40% W/W.
